(19) United States
(12) Patent Application Publication
(10) Pub. No.: US 2007/0212547 A1
(43) Pub. Date: Sep. 13, 2007

(54) Method of powder coating medical devices

(75) Inventors: Gerald Fredrickson, Westford, MA (US); M. J. Timm, Littleton, MA (US)

Correspondence Address: KENYON & KENYON LLP, 1500 K STREET N.W., SUITE 700, WASHINGTON, DC, 20005, US

(73) Assignee: BOSTON SCIENTIFIC SCIMED, INC.

(21) Appl. No.: 11/369,992

(22) Filed: Mar. 8, 2006

(51) Int. Cl. A61L 33/00; B05D 1/12; B05D 3/10; B32B 27/00

(52) U.S. Cl. 428/411.1; 427/2.1; 427/180; 427/337

(57) ABSTRACT

A method of creating a polymer coating on a medical device by powder coating the medical device with a powder material comprising a polymer and applying a solvent onto the powder coating to coalesce the powder coating into a continuous polymer film. A therapeutic agent may be mixed into the powder material, mixed into the coalescing solvent, or incorporated into the resulting polymer film. Also provided is a medical device having a polymer coating wherein the polymer coating is created according to the methods of the present invention.

### TECHNICAL FIELD

The present invention relates to methods of coating medical devices with a polymer coating.

### BACKGROUND

Many implantable medical devices are coated with a therapeutic agent or drug that acts to improve the effectiveness of the device. One such example of a drug-coated implantable medical device is a coronary stent. Coronary stents are tubular structures formed in a mesh-like pattern that are designed to be inserted into a coronary artery across an area of blockage that has been opened by an angioplasty procedure. The stent serves as a permanent scaffolding for the newly widened coronary artery.

In many instances, however, the stented artery becomes narrowed again in a process known as restenosis, which results from vessel wall injury, local inflammation, and tissue remodeling following the balloon angioplasty and stenting. Therefore, many coronary artery stents are coated with a drug, such as paclitaxel or other therapeutic agent, that acts to inhibit the processes that cause restenosis.

Stents can be coated by various conventional coating processes, such as spray coating, electrostatic spraying, or dip coating. These prior processes have various advantages and disadvantages. For example, spray coating methods often have low transfer efficiencies because much of the coating solution is lost in excessive overspraying. Transfer efficiencies are important as some coating materials are expensive, such as therapeutic agents, drugs and polymers. Also, certain spray coating methods, such as gas-assisted spray coating, can impart a high degree of shear to the coating solution, resulting in damage to shear sensitive coating materials.

### SUMMARY OF THE INVENTION

The present invention includes embodiments directed to a method of powder coating medical devices. In one embodiment, a medical device is coated with a powder coating wherein the powder coating comprises a polymer. A solvent is applied to the powder coating to coalesce the polymer in the powder coating into a continuous polymer film. The solvent may be sprayed onto the powder coating. The solvent may be removed by evaporation at room temperature, or under low heat, or under vacuum drying. A therapeutic agent may be mixed into the powder coating material, mixed into the coalescing solvent, or incorporated into the resulting polymer film.

In another embodiment, a medical device is coated with a powder coating wherein the powder coating comprises a polymer; a solvent is applied to the powder coating; and heat is applied to the powder coating. The application of low heat to the powder coating may assist in coalescing the polymer, evaporating the solvent, or both.

The present invention also includes embodiments directed to medical devices coated with polymer films formed by the coating methods of the present invention.

### DETAILED DESCRIPTION

The present invention includes embodiments directed to a method of powder coating a medical device. The powder coating material used in this invention comprises polymers which may be available in powder form, or a polymer in solution may be converted into a powder formulation by various methods known in the art, including spray drying, pelletization, micronization, and cryogenic cooling with grinding. The powder coating material may be in the form of a fine powder with particle sizes suitable for use in conventional powder coating processes. The powder coating material can be applied onto the medical device by various known methods including the use of fluid beds, electrostatic fluid beds, and electrospray guns (including corona-charged and tribo-charged guns). The thickness of the coating will vary depending upon the medical device and desired function of the coating.

Powder coating may be restricted to certain portions of the medical device by masking techniques that are known in the art. In conventional masking techniques, certain areas of the medical device may be physically covered or blocked to prevent powder deposition. In electrostatic masking techniques, a charged body is used to redirect or repel the powder coating material. In one example, such masking techniques may be used to restrict the powder coating to the outer diameter of a stent.

The polymers used in the present invention are those having the desired biological, chemical, physical, mechanical, or pharmacologic properties for its use in the coating of medical devices and implantable medical devices in particular. For example, in drug-eluting stents, the polymers used can be styrene-isobutylene block copolymers such as styrene-isobutylene-styrene tri-block copolymers (SIBS) and other block copolymers such as styrene-ethylene/butylene-styrene (SEBS). The polymers may have a glass transition temperature (T_{g}) in the range of −120° C. to 200° C. in order to facilitate low temperature curing. Where room temperature curing is desired, the polymers may have a T_{g} in the range of 20° C. to 200° C.

A solvent is then applied to the powder coating by various methods known in the art, including spraying, electrostatic spraying, dip coating, and the like. An electrostatic fine mist spray of solvent may be used in order to minimize disturbance to the powder layer. The solvent coalesces the polymer in the powder coating into a continuous polymer film. The solvent may accomplish this by dissolving, solubilizing, or emulsifying the polymer, or otherwise allowing the polymer chains to flow together at a temperature below its T_{g} to yield a continuous polymer film. The coalescence may occur at room temperature or under low heat. In embodiments where a therapeutic agent is mixed into the powder coating material or solvent, the heat used to coalesce the polymer is sufficiently low that the therapeutic agent does not significantly degrade. For example, in a coronary stent coated with a powder coating mixture of SIBS and paclitaxel, low heat in the range of 30° C. to 75° C. for a duration of one to ten hours would yield a continuous polymer film with little or no drug degradation.

Various solvents that are capable of coalescing the polymer particles into a continuous film are suitable for use in the solvent coalescing step. Solvents that allow good flow of the polymer chains at low temperatures may be used, including solvents that dissolve the polymer. In certain embodiments of the present invention where a therapeutic agent is mixed into the solvent, solvents are further selected for their ability to dissolve or not dissolve the drug, depending upon the desired drug release characteristics of the resulting polymer film. In the example of a coronary stent coated with paclitaxel and SIBS, tetrahydrafuran (THF) may be preferred for its ability to dissolve both the drug and polymer. In other instances, however, it may be desirable to select a solvent that does not dissolve the drug. For example, where a particulate, non-homogenous coating of paclitaxel is desired, THF blended with a solvent in which paclitaxel is not soluble, such as toluene or xylene, would be preferred. With the appropriate selection of solvents and coalescing conditions such as temperature, one of skill in the art would be able to create polymer coatings with varying properties, including ones that have the desired drug release characteristics. Also, one of skill in the art could use the method of the present invention to closely replicate the stent coatings that are formed by conventional spray coating processes.

Simultaneous with or after the step of coalescing the polymer, the solvent is removed from the coating by evaporation. Low heat that can be applied to assist in coalescing the powder coating may also be used to serve the purpose of assisting in solvent evaporation. Vacuum drying could also be used to assist in evaporating the solvent. In the example of a SIBS/paclitaxel-coated stent, one to ten hours of low heat in the range of 30° C. to 75° C. under vacuum would be sufficient to fully remove the solvent. Because there is an inverse relationship between drying duration and temperature, shorter drying times could be achieved at higher temperatures, or alternatively, lower temperatures could be used with longer drying times. With the appropriate selection of drying conditions, including duration and application of heat or vacuum, one of skill in the art would be able to create coatings with varying properties.

In certain embodiments of the present invention, a therapeutic agent is dispersed within the resulting polymer coating. The therapeutic agent may be added at various steps in the method of the present invention. In one embodiment, the therapeutic agent may be introduced into the powder coating material. The therapeutic agent may be available in powder form, or may be converted into a powder formulation by various known methods such as spray drying, pelletization, micronization, and cryogenic cooling with grinding, and then mixed with the polymer powder. Alternatively, the polymer and drug may be mixed in a solution, suspension, or dispersion, and the combined mixture may be converted into a powder formulation.

In other embodiments, the therapeutic agent may be mixed into the solvent that is used to coalesce the powder coating. The solvent may or may not dissolve the drug, depending upon the desired drug release characteristics of the resulting polymer film. In still other embodiments, the therapeutic agent may be incorporated into the polymer film by conventional methods such as spray coating, dip coating, vacuum impregnation, or electrophoretic transfer, as a subsequent step after the polymer film is created.

The powder coating method of the present invention may also be applied repetitively, or in combination with conventional spray coating techniques, which may, in some cases, result in the creation of multiple discrete layers. For example, a first coating can be applied to a medical device by conventional techniques, followed by a second coating applied over the first coating using the powder coating method of the present invention. Alternatively, a first coating can be applied by the powder coating method of the present invention, followed by a second coating applied over the first coating using conventional techniques. With these techniques, two or more discrete layers can be created where the outer layers can be used to control the diffusion rate of therapeutic agent released from the inner layers.

Coating medical devices by powder coating methods in accordance with the present invention offers several advantages over other types of coating methods. In general, powder coating methods have a very high transfer efficiency, approaching nearly 100% in some cases. This is because the powder coating material is dry and any overspray can readily be retrieved and reused. This advantage is particularly beneficial where expensive polymers and/or drugs are being applied to medical devices.

In general, powder coating equipment is also less expensive and less costly to maintain than other conventional spray coating equipment. Powder coating further has the advantages of not applying damaging shear forces to fragile coating materials and being suitable for use with coating materials that are not easily soluble in typical spray coating solvents.

The use of solvents to coalesce the polymer of the powder coating material also offers some advantages. The method avoids the use of high temperature curing, which may not be suitable for heat sensitive drugs or polymers used in medical device coatings. Also, the method avoids the use of plasticizers, which allows for lower temperature curing, but which may not be biocompatible and would require regulatory approval for use in implantable medical devices.

The medical device of the present invention is not limited to the coronary stents in the disclosed embodiments. Non-limiting examples of other medical devices that can be used with the coating methods of the present invention include catheters, guide wires, balloons, filters (e.g., vena cava filters), stents, stent grafts, vascular grafts, intraluminal paving systems, pacemakers, electrodes, leads, defibrillators, joint and bone implants, spinal implants, vascular access ports, intra-aortic balloon pumps, heart valves, sutures, artificial hearts, neurological stimulators, cochlear implants, retinal implants, and other devices that can be used in connection with therapeutic coatings. Such medical devices are implanted or otherwise used in body structures, cavities, or lumens such as the vasculature, gastrointestinal tract, abdomen, peritoneum, airways, esophagus, trachea, colon, rectum, biliary tract, urinary tract, prostate, brain, spine, lung, liver, heart, skeletal muscle, kidney, bladder, intestines, stomach, pancreas, ovary, uterus, cartilage, eye, bone, and the like.

The therapeutic agent in the powder coating material, or coalescing solvent, or the polymer film coating the medical device may be any pharmaceutically acceptable agent such as a non-genetic therapeutic agent, a biomolecule, a small molecule, or cells. The therapeutic agent may be available in powder form, or may be converted into a powder formulation by any known method including cryogenic cooling with grinding, drying, micronizing, or spraying onto the medical device and drying.

Exemplary non-genetic therapeutic agents include anti-thrombogenic agents such heparin, heparin derivatives, prostaglandin (including micellar prostaglandin E1), urokinase, and PPack (dextrophenylalanine proline arginine chloromethylketone); anti-proliferative agents such as enoxaparin, angiopeptin, sirolimus (rapamycin), tacrolimus, everolimus, zotarolimus, monoclonal antibodies capable of blocking smooth muscle cell proliferation, hirudin, and acetylsalicylic acid; anti-inflammatory agents such as dexamethasone, rosiglitazone, prednisolone, corticosterone, budesonide, estrogen, estrodiol, sulfasalazine, acetylsalicylic acid, mycophenolic acid, and mesalamine; anti-neoplastic/anti-proliferative/anti-mitotic agents such as paclitaxel, epothilone, cladribine, 5-fluorouracil, methotrexate, doxorubicin, daunorubicin, cyclosporine, cisplatin, vinblastine, vincristine, epothilones, endostatin, trapidil, halofuginone, and angiostatin; anti-cancer agents such as antisense inhibitors of c-myc oncogene; anti-microbial agents such as triclosan, cephalosporins, aminoglycosides, nitrofurantoin, silver ions, compounds, or salts; biofilm synthesis inhibitors such as non-steroidal anti-inflammatory agents and chelating agents such as ethylenediaminetetraacetic acid, O,O′-bis (2-aminoethyl) ethyleneglycol-N,N,N′,N′-tetraacetic acid and mixtures thereof; antibiotics such as gentamycin, rifampin, minocyclin, and ciprofloxacin; antibodies including chimeric antibodies and antibody fragments; anesthetic agents such as lidocaine, bupivacaine, and ropivacaine; nitric oxide; nitric oxide (NO) donors such as linsidomine, molsidomine, L-arginine, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, warfarin sodium, Dicumarol, aspirin, prostaglandin inhibitors, platelet aggregation inhibitors such as cilostazol and tick antiplatelet factors; vascular cell growth promotors such as growth factors, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vascoactive mechanisms; inhibitors of heat shock proteins such as geldanamycin; angiotensin converting enzyme (ACE) inhibitors; beta-blockers; βAR kinase (βARK) inhibitors; phospholamban inhibitors; protein-bound particle drugs such as ABRAXANE™; and any combinations and prodrugs of the above.

Exemplary biomolecules include peptides, polypeptides and proteins; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Nucleic acids may be incorporated into delivery systems such as, for example, vectors (including viral vectors), plasmids or liposomes.

Non-limiting examples of proteins include serca-2 protein, monocyte chemoattractant proteins (MCP-1) and bone morphogenic proteins (“BMP's”), such as, for example, BMP-2, BMP-3, BMP-4, BMP-5, BMP-6 (VGR-1), BMP-7 (OP-1), BMP-8, BMP-9, BMP-10, BMP-11, BMP-12, BMP-13, BMP-14, BMP-15. Preferred BMP's are any of BMP-2, BMP-3, BMP-4, BMP-5, BMP-6, and BMP-7. These BMPs can be provided as homodimers, heterodimers, or combinations thereof, alone or together with other molecules. Alternatively, or in addition, molecules capable of inducing an upstream or downstream effect of a BMP can be provided. Such molecules include any of the “hedghog” proteins, or the DNA's encoding them. Non-limiting examples of genes include survival genes that protect against cell death, such as anti-apoptotic Bcl-2 family factors and Akt kinase; serca 2 gene; and combinations thereof. Non-limiting examples of angiogenic factors include acidic and basic fibroblast growth factors, vascular endothelial growth factor, epidermal growth factor, transforming growth factors α and β, platelet-derived endothelial growth factor, platelet-derived growth factor, tumor necrosis factor α, hepatocyte growth factor, and insulin-like growth factor. A non-limiting example of a cell cycle inhibitor is a cathespin D (CD) inhibitor. Non-limiting examples of anti-restenosis agents include p15, p16, p18, p19, p21, p27, p53, p57, Rb, nFkB and E2F decoys, thymidine kinase and combinations thereof and other agents useful for interfering with cell proliferation.

Exemplary small molecules include hormones, nucleotides, amino acids, sugars, and lipids and compounds have a molecular weight of less than 100 kD.

Exemplary cells include stem cells, progenitor cells, endothelial cells, adult cardiomyocytes, and smooth muscle cells. Cells can be of human origin (autologous or allogenic) or from an animal source (xenogenic), or genetically engineered. Non-limiting examples of cells include side population (SP) cells, lineage negative (Lin⁻) cells including Lin⁻CD34⁻, Lin-CD34⁺, Lin⁻cKit⁺, mesenchymal stem cells including mesenchymal stem cells with 5-aza, cord blood cells, cardiac or other tissue derived stem cells, whole bone marrow, bone marrow mononuclear cells, endothelial progenitor cells, skeletal myoblasts or satellite cells, muscle derived cells, go cells, endothelial cells, adult cardiomyocytes, fibroblasts, smooth muscle cells, adult cardiac fibroblasts +5-aza, genetically modified cells, tissue engineered grafts, MyoD scar fibroblasts, pacing cells, embryonic stem cell clones, embryonic stem cells, fetal or neonatal cells, immunologically masked cells, and teratoma derived cells.

Any of the therapeutic agents may be combined to the extent such combination is biologically compatible.

The polymers used in the present invention may be available in powder form, or converted into a powder formulation by any method known in the art. The polymers may be biodegradable or non-biodegradable. Non-limiting examples of suitable non-biodegradable polymers include polystrene; polystyrene maleic anhydride; poly(methylmethacrylate-butylacetate-methylmethacrylate); polyisobutylene copolymers; styrene-isobutylene block copolymers such as styrene-isobutylene-styrene tri-block copolymers (SIBS) and other block copolymers such as styrene-ethylene/butylene-styrene (SEBS); polyvinylpyrrolidone including cross-linked polyvinylpyrrolidone; polyvinyl alcohols, copolymers of vinyl monomers such as EVA; polyvinyl ethers; polyvinyl aromatics; polyethylene oxides; polyesters including polyethylene terephthalate; polyamides; polyacrylamides; polyethers including polyether sulfone; polyalkylenes including polypropylene, polyethylene and high molecular weight polyethylene; polyurethanes; polycarbonates, silicones; siloxane polymers; cellulosic polymers such as cellulose acetate; polymer dispersions such as polyurethane dispersions (BAYHYDROL®); squalene emulsions; and mixtures and copolymers of any of the foregoing.

Non-limiting examples of suitable biodegradable polymers include polycarboxylic acid, polyanhydrides including maleic anhydride polymers; polyorthoesters; poly-amino acids; polyethylene oxide; polyphosphazenes; polylacetic acid, polyglycolic acid and copolymers and mixtures thereof such as poly(L-lacetic acid) (PLLA), poly(D,L,-lactide), poly(lacetic acid-co-glycolic acid), 50/50 (DL-lactide-co-glycolide); polydioxanone; polypropylene fumarate; polydepsipeptides; polycaprolactone and co-polymers and mixtures thereof such as poly(D,L-lactide-co-caprolactone) and polycaprolactone co-butylacrylate; polyhydroxybutyrate valerate and blends; polycarbonates such as tyrosine-derived polycarbonates and arylates, polyiminocarbonates, and polydimethyltrimethylcarbonates; cyanoacrylate; calcium phosphates; polyglycosaminoglycans; macromolecules such as polysaccharides (including hyaluronic acid; cellulose, and hydroxypropylmethyl cellulose; gelatin; starches; dextrans; alginates and derivatives thereof), proteins and polypeptides; and mixtures and copolymers of any of the foregoing. The biodegradable polymer may also be a surface erodable polymer such as polyhydroxybutyrate and its copolymers, polycaprolactone, polyanhydrides (both crystalline and amorphous), maleic anhydride copolymers, and zinc-calcium phosphate.

A variety of solvents may be used as the coalescing solvent in the present invention including methanol, ethanol, N-propanol, isopropanol, butoxydiglycol, butoxyethanol, butoxyisopropanol, butoxypropanol, n-butyl alcohol, t-butyl alcohol, butylene glycol, butyl octanol, diethylene glycol, dimethoxydiglycol, dimethyl ether, dipropylene glycol, ethoxydiglycol, ethoxyethanol, ethyl hexane diol, glycol, hexane diol, 1,2,6-hexane triol, hexyl alcohol, hexylene glycol, isobutoxy propanol, isopentyl diol, 3-methoxybutanol, methoxydiglycol, methoxyethanol, methoxyisopropanol, methoxymethylbutanol, methoxy PEG-10, methylal, methyl hexyl ether, methyl propane diol, neopentyl glycol, PEG-4, PEG-6, PEG-7, PEG-8, PEG-9, PEG-6-methyl ether, pentylene glycol, PPG-7, PPG-2-buteth-3, PPG-2 butyl ether, PPG-3 butyl ether, PPG-2 methyl ether, PPG-3 methyl ether, PPG-2 propyl ether, propane diol, propylene glycol, propylene glycol butyl ether, propylene glycol propyl ether, tetrahydrofuran, trimethyl hexanol, phenol, benzene, toluene, xylene; as well as water, if necessary in mixture with dispersants and mixtures of the above-named substances.

While the various elements of the disclosed invention are described and/or shown in various combinations and configurations, which are exemplary, other combinations and configurations, including more, less or only a single embodiment, are also within the spirit and scope of the present invention.

What is claimed is:

1. A method of coating a medical device, comprising the steps of:
(a) applying a powder coating to a medical device, wherein the powder coating comprises a polymer;
(b) applying a solvent to the powder coating, wherein the solvent coalesces the polymer; and
(c) removing the solvent.

2. The method of claim 1, wherein the medical device is a stent.

3. The method of claim 1, wherein the step of applying a powder coating is performed by an electrostatic powder coating process.

4. The method of claim 1, wherein the step of applying a solvent to the powder coating comprises spraying the solvent onto the powder coating.

5. The method of claim 1, further comprising the step of selecting a solvent that dissolves, solubilizes, or emulsifies the polymer.

6. The method of claim 1, further comprising the step of selecting a solvent that allows the polymer to flow at a temperature below the T_{g} of the polymer.

7. The method of claim 1, wherein the step of removing the solvent comprises evaporating the solvent.

8. The method of claim 7, wherein the step of evaporating the solvent comprises applying low heat to the powder coating.

9. The method of claim 7, wherein the step of evaporating the solvent comprises vacuum drying the solvent.

10. The method of claim 1, wherein the powder coating further comprises a therapeutic agent.

11. The method of claim 1, wherein the solvent further comprises a therapeutic agent.

12. The method of claim 1, further comprising the step of incorporating a therapeutic agent into the polymer film.

13. A method of coating a medical device, comprising the steps of:
(a) applying a powder coating to a medical device, wherein the powder coating comprises a polymer;
(b) applying a solvent to the powder coating; and
(c) applying low heat to the powder coating.

14. The method of claim 13, wherein applying low heat to the powder coating causes the polymer to coalesce.

15. The method of claim 13, wherein the step of applying a powder coating is performed by an electrostatic powder coating process.

16. The method of claim 13, wherein the step of applying a solvent to the powder coating comprises spraying the solvent onto the powder coating.

17. The method of claim 13, further comprising the step of selecting a solvent that dissolves, solubilizes, or emulsifies the polymer.

18. The method of claim 13, further comprising the step of selecting a solvent that allows the polymer to flow at a temperature below the T_{g} of the polymer.

19. The method of claim 13, further comprising the step of removing the solvent.

20. The method of claim 19, wherein the step of removing the solvent comprises evaporating the solvent.

21. The method of claim 20, wherein the step of evaporating the solvent comprises applying low heat to the powder coating.

22. The method of claim 20, wherein the step of evaporating the solvent comprises vacuum drying the solvent.

23. The method of claim 13, wherein the powder coating further comprises a therapeutic agent.

24. The method of claim 13, wherein the solvent further comprises a therapeutic agent.

25. The method of claim 13, further comprising the step of incorporating a therapeutic agent into the polymer film.

26. A medical device having a polymer coating, wherein the polymer coating is formed by the method of claim 1.

27. A medical device having a polymer coating, wherein the polymer coating is formed by the method of claim 13.
